(19) United States
(12) Patent Application Publication
(10) Pub. No.: US 2012/0157382 A1
(43) Pub. Date: Jun. 21, 2012

(54) PHARMACEUTICAL GLP-1 COMPOSITIONS HAVING AN IMPROVED RELEASE PROFILE

(76) Inventors: Siegfried Krimmer, Steinen (DE); Marc Lindenberg, Loerrach (DE); Annie Miesch, Riespach (FR); Robert Mueller, Basel (CH); Marco Mumenthaler, Muttenz (CH)

(21) Appl. No.: 13/325,094

(22) Filed: Dec. 14, 2011

(30) Foreign Application Priority Data
Dec. 21, 2010 (EP) 10196161.3

(51) Int. Cl. A61K 38/26; A61P 3/04; A61P 25/28; A61P 15/00; A61P 9/04; A61P 9/10; A61P 9/00; A61P 3/10; A61P 13/12

(52) U.S. Cl. 514/11.7

(57) ABSTRACT

This invention relates to sustained release liquid pharmaceutical compositions containing a peptide analogue of glucagon-like peptide-1 or salts thereof having an improved release profile. The invention also relates to methods for preparing such compositions.

### PRIORITY TO RELATED APPLICATION(S)

This application claims the benefit of European Patent Application No. 10196161.3, filed Dec. 21, 2010, which is hereby incorporated by reference in its entirety.

### FIELD OF THE INVENTION

The invention relates to sustained release liquid pharmaceutical compositions comprising a peptide analogue of glucagon-like peptide-1 (GLP-1) or salts thereof having an improved release profile and to methods for preparing such compositions.

More specifically, the invention relates to improved sustained release liquid pharmaceutical compositions comprising [Aib^{8,35}]hGLP-1(7-36)NH₂, a GLP-1 analogue with the amino acid sequence according to SEQ ID No. 1:

His-Aib-Glu-Gly-Thr-Phe-Thr-Ser-Asp-Val-Ser-Ser-Tyr-Leu-Glu-Gly-Gln-Ala-Ala-Lys-Glu-Phe-Ile-Ala-Trp-Leu-Val-Lys-Aib-Arg-NH₂,

wherein 26 of these amino acids are in the natural L configuration while four are not chiral. The naturally occurring amino acid residues in position 8 (Ala) and 35 (Gly) have been substituted by α-aminoisobutyric acid (Aib). [Aib^{8,35}]hGLP-1(7-36)NH₂ is also known as taspoglutide.

The improved sustained release liquid pharmaceutical compositions further comprise a divalent metal salt, preferably zinc chloride, wherein the molar ratio range of the peptide analogue to the divalent metal is 1.5 to 1, a liquid such as water, and optionally acetic acid and/or an acetate salt, wherein the molar ratio range of the acetic acid and/or acetate salt to the peptide is less than 3.2 to 1, and are characterized in that the final pH of the composition in the range of 4.4 to 4.6 is adjusted by addition of hydrochloric acid.

### BACKGROUND OF THE INVENTION

GLP-1 is secreted in the body from intestinal L cells as a gut hormone. The biologically active forms of GLP-1 are GLP-1 (7-37) and GLP-1 (7-36)NH₂. Those peptides result from selective cleavage of proglucagon. Once in the circulation, GLP-1 has a half life of less than 2 minutes, due to rapid degradation by the enzyme dipeptidylase-4 (DPP-4). It is a potent antihyperglycemic hormone, inducing glucose-dependent stimulation of insulin secretion while suppressing glucagon secretion. GLP-1 and analogues thereof are thus useful in subjects having non-insulin dependent diabetes as well as for the treatment of gestational diabetes mellitus

In addition, there are a number of therapeutic uses, for which GLP-1 and analogues thereof have been suggested, including enhancing neuroprotection, and/or alleviating a symptom of a disease or disorder of the central nervous system, e.g., through modulation of neurogenesis, and e.g., Parkinson's Disease, Alzheimer's Disease, Huntington's Disease, ALS, stroke, ADD, and neuropsychiatric syndromes), preventing beta-cell deterioration, stimulation of beta-cell proliferation, treating obesity by suppressing appetite and inducing satiety, reducing the morbidity and/or mortality associated with myocardial infarction and stroke, treating acute coronary syndrome characterized by an absence of Q-wave, myocardial infarction, attenuating postsurgical catabolic changes, treating hibernating myocardium or diabetic cardiomyopathy, suppressing plasma blood levels of norepinepherine, increasing urinary sodium excretion, decreasing urinary potassium concentration, treating conditions or disorders associated with toxic hypervolemia, e.g., renal failure, congestive heart failure, nephrotic syndrome, cirrhosis, pulmonary edema, and hypertension, inducing an inotropic response and increasing cardiac contractility, treating polycystic ovary syndrome, treating respiratory distress, improving nutrition via a non-alimentary route, i.e., via intravenous, subcutaneous, intramuscular, peritoneal, or other injection or infusion, treating nephropathy, treating left ventricular systolic dysfunction, e.g., with abnormal left ventricular ejection fraction, inhibiting antro-duodenal motility, e.g., for the treatment or prevention of gastrointestinal disorders such as diarrhea, post-operative dumping syndrome and irritable bowel syndrome, and as premedication endoscopic procedures, treating critical illness polyneuropathy (CIPN) and systemic inflammatory response syndrome (SIRS), modulating triglyceride levels and treating dyslipidemia, treating organ tissue injury caused by reperfusion of blood flow following ischemia and treating coronary heart disease risk factor (CHDRF) syndrome.

The therapeutic potential of GLP-1 is, however, limited in view of its metabolic instability, having a plasma half-life (t_{1/2}) of only 1 to 2 min. in vivo. A number of attempts have been taken to improve the therapeutic potential of GLP-1 and its analogues through improvements in formulation.

European patent publication No. EP 0 619 322 A2 describes the preparation of micro-crystalline forms of GLP-1(7-37)OH by mixing solutions of the protein in pH 7 to 8.5 buffer with certain combinations of salts and low molecular weight polyethylene glycols (PEG). Similarly, biodegradable polymers, such as poly(lactic-co-glycolic acid) (PLGA) and a triblock copolymer of poly [(dl-lactide-co-glycolide)-β-ethylene glycol-/3-(-lactide-co-glycolide)], have also been suggested for use in sustained delivery formulations of GLP-1 peptides. However the use of such biodegradable polymers has been disfavored in the art since these polymers generally have poor solubility in water and require water-immiscible organic solvents, e.g., methylene chloride, and/or harsh preparation conditions during manufacture. Such organic solvents and/or harsh preparation conditions are considered to increase the risk of inducing conformational change of the peptide or protein of interest, resulting in decreased structural integrity and compromised biological activity.

There is a need for GLP-1 formulations which are more easily and reliably manufactured, and which are more easily and reproducibly administered to a patient Sustained release liquid pharmaceutical compositions comprising [Aib^{8,35}]hGLP-1(7-36)NH₂, a liquid, zinc and/or zinc chloride and an acetate and/or acetic acid, wherein the molar ratio range of the peptide to zinc is approximately 6:1 to 1:1 and the molar ratio range of the acetate and/or acetic acid to the peptide is approximately 1:1 to 6:1, and wherein the pH of said pharmaceutical composition is within the range of 4 to 5, are disclosed in WO 2010/089672. Subcutaneous administration of sustained release formulations of [Aib^{8,35}]hGLP-1(7-36)NH₂ with a divalent metal salt such as zinc salts aims for the formation of a solidified depot under the skin and a sustained release of [Aib^{8,35}]hGLP-1(7-36)NH₂. These compositions have the advantage of forming a solidified depot under the skin when injected subcutaneously and are thus able to release the peptide over a time range of at least one week to two weeks. However, the plasma profiles obtained after a single (s.c.) administration to dogs (see FIGS. 1 to 3, 5 and 6 of WO 2010/089672) clearly show, that the compositions are characterized by an initial burst, i.e. by high initial plasma concentration shortly after administration, which is assumed to be responsible for the occurrence of dose-related, side effects such as vomiting.

The object of the present invention therefore is to modify the original pharmaceutical composition of [Aib^{8,35}]hGLP-1(7-36)NH₂ in order to obtain a pharmaceutical composition with an improved sustained release profile in which the initial burst effect (i.e. the initial plasma concentration) is reduced in order to eliminate unwanted side effects.

It was found that this object could be reached with the sustained release liquid pharmaceutical composition as outlined below.

### SUMMARY OF THE INVENTION

The present invention relates to a sustained release liquid pharmaceutical composition having a pH in the range of 4.4 to 4.6 comprising:

a liquid solvent,

a peptide analogue having the formula

[Aib^{8,35}]hGLP-1(7-36)NH₂(I), and

a divalent metal salt, wherein the molar ratio range of the peptide analogue to the divalent metal is 1.5 to 1,

characterized in that the final pH in the range of 4.4 to 4.6 is adjusted by addition of hydrochloric acid.

### DETAILED DESCRIPTION OF THE INVENTION

The present invention relates to a sustained release liquid pharmaceutical composition having a pH in the range of 4.4 to 4.6 comprising:

a liquid solvent,

a peptide analogue having the formula

[Aib^{8,35}](7-36)NH₂(I),

a divalent metal salt, wherein the molar ratio range of the peptide analogue to the divalent metal is 1.5 to 1, and

optionally acetic acid and/or an acetate salt, wherein the molar ratio range of the acetic acid and/or acetate salt to the peptide is less than 3.2 to 1,

characterized in that the final pH in the range of 4.4 to 4.6 is adjusted by addition of hydrochloric acid.

In particular, the invention relates to a sustained release liquid pharmaceutical composition having a pH in the range of 4.4 to 4.6 comprising:

water,

a peptide analogue having the formula

[Aib^{8,35}]hGLP-1(7-36)NH₂(I),

a divalent metal salt, wherein the molar ratio range of the peptide analogue to the divalent metal is 1.5 to 1, and

optionally acetic acid and/or an acetate salt, wherein the molar ratio range of the acetic acid and/or acetate salt to the peptide is less than 3.2 to 1,

characterized in that the final pH in the range of 4.4 to 4.6 is adjusted by addition of hydrochloric acid.

In particular, the acetate salt is sodium acetate trihydrate.

The divalent metal salt is selected from the group consisting of zinc cloride (ZnCl₂), zinc acetate dihydrate, copper chloride (CuCl2) and copper acetate. More particularly, the divalent metal salt is zinc chloride.

Thus, the present invention relates to a sustained release liquid pharmaceutical composition having a pH in the range of 4.4 to 4.6 comprising:

water,

a peptide analogue having the formula

[Aib^{8,35}]hGLP-1(7-36)NH₂(I),

zinc chloride, wherein the molar ratio range of the peptide analogue to zinc chloride is 1.5 to 1, and

optionally acetic acid and or sodium acetate trihydrate, wherein the molar ratio range of the acetic acid and/or acetate salt to the peptide is less than 3.2 to 1,

characterized in that the final pH in the range of 4.4 to 4.6 is adjusted by addition of hydrochloric acid.

In particular, the concentration of zinc chloride is 2.72 mg/ml (20 mM).

In particular, the concentration of the peptide analogue [Aib^{8,35}]hGLP-1(7-36)NH₂ is 100 mg/ml (30 mM).

The invention further relates to a liquid pharmaceutical composition as defined above, wherein the maximum concentration of acetic acid and/or sodium acetate trihydrate is 95 mM.

In particular, the present invention relates to a sustained release liquid pharmaceutical composition having a pH in the range of 4.4 to 4.6 comprising:

water,

[Aib^{8,35}]hGLP-1(7-36)NH₂ in a concentration of 100 mg/ml (30 mM)

zinc chloride in a concentration of 2.72 mg/ml (20 mM), and

acetic acid and/or an acetate salt, wherein acetic acid is added in a concentration range of 0 mM to 75 mM,

characterized in that the final pH in the range of 4.4 to 4.6 is adjusted by addition of hydrochloric acid.

In particular, acetic acid is added in a concentration range of 0 mM to 47.5 mM. More particularly, acetic acid is added in a concentration of 20 mM.

The invention further relates to a liquid pharmaceutical composition as defined above, wherein the composition further comprises sodium chloride in a concentration range of 0 mM to 50 mM.

The pharmaceutical compositions according to the invention are further characterized in that they are stable at a temperature of 5° C. for a period of at least one year.

The term “sustained release” as used herein means a release which results in a measurable serum level of the biologically active peptide analogue of formula I, for a period of at least 1 week and more preferably for a period of at least 2 weeks.

Thus, the invention relates to pharmaceutical compositions, wherein the composition is formulated such that [Aib^{8,35}]hGLP-1(7-36)NH₂ is released within the human subject for at least approximately 1 week, preferably 2 weeks.

The sustained release profile and ability of the pharmaceutical compositions of the present invention to show a reduced initial burst effect has been investigated by using in vitro precipitation and dissolution assays.

In the precipitation assay a certain amount of the pharmaceutical composition is added to a phosphate buffer solution of pH 7.4 (see Example 3.1). The mixture is then centrifuged and the remaining dissolved peptide analogue of formula I (([Aib^{8,35}]hGLP-1(7-36)NH₂) in the supernatent is measured by analytical HPLC, A high amount of peptide in solution corresponds to a strong initial burst effect.

In particular, the pharmaceutical compositions according to the invention are those, wherein less than 2.5% of the dose of the peptide analogue of formula I ([Aib^{8,35}]hGLP-1(7-36)NH₂) can be detected in the supernatant after precipitation in phosphate buffer pH 7.4 and subsequent centrifugation.

In the dissolution assay, the release of the peptide from a precipitated depot to a continuous flow of release medium can be studied (Example 3.2). 0.2 ml of the pharmaceutical composition are added into a cell filled with glass beadlets. The release medium is a Hanks balanced salt solution (HBSS) modified with 25 mM HEPES (4-(2-Hydroxyethyl)piperazine-1-ethanesulfonic acid) buffering agent at pH 7.4 which flows through the cell. Samples are collected after 5, 10, 15, 30 and 60 min and their content of dissolved/released peptide is analyzed.

Particularly interesting pharmaceutical compositions according to the invention are those, wherein less than 0.015 mg/min of the peptide analogue of formula I are released after 5 min during dissolution in modified HBSS buffer pH 7.4.

The present invention further relates to a method for preparing the pharmaceutical compositions as defined above, comprising the steps of

a) combining the liquid solvent and acetic acid and/or acetate salt to obtain an acidified solution,
b) dissolving the peptide analogue in the acidified solution,
c) adding zinc chloride and optionally sodium chloride; and
d) adjusting the pH of the final solution with hydrochloric acid to be in the range of 4.4 to 4.6. Zinc chloride and sodium chloride can be added as solutions or they can be added in the solid state and dissolved in the acidified solution.

More particularly, the invention is concerned with the method as described above further comprising the steps of

e) sterile filtrating the composition resulting from step d), and
f) filling a container with the composition.

As described above, the pharmaceutical compositions as described above are specifically suitable for subcutaneous administration, i.e. for injection into the skin. The invention thus also relates to a pharmaceutical composition according to the invention, wherein it is kept in a container, for example a pre-filled syringe.

Thus, the invention also relates to a pre-filled syringe containing a pharmaceutical composition according to the invention. In particular, the pre-filled syringe contains 0.2 ml of the pharmaceutical composition according to the invention.

The pharmaceutical compositions according to the invention are especially suitable for use in the treatment of Type 2 diabetes or gestational diabetes. They are also useful for the treatment of other metabolic diseases such as obesity, central nervous system diseases such as Alzheimer's disease, renal failure and cardiovascular diseases such as congestive heart failure, myocardial infarction, stroke and acute coronary syndrome, polycystic ovary syndrome nephrotic syndrome.

### EXAMPLES

### Example 1

### Preparation of [Aib^{8,35}]hGLP-1(7-36)NH₂(Drug Substance)

### Preparation of the Peptide:

The crude peptide [Aib^{8,35}]hGLP-1(7-36)NH₂ can be prepared according to the methods described in WO 2007/147816 and WO 2009/074483 by producing three fragments and coupling these fragments in solution.

### RP-HPLC Purification:

Purification of the crude peptide was performed on a RP (reversed phase) stationary phase. Thus, the sorbent is RP material such as silica gel (e.g. Kromasil 100-16-C18) or acrylic ester macroreticular adsorbent (e.g. Amberchrom CG71M). The purification involved a 1^{st} pass chromatographic purification at a pH of approximately 2, followed by a 2^{nd} pass at a pH of approximately 9.

### 1^{st} Chromatography:

Crude [Aib^{8,35}]hGLP-1(7-36)NH₂ was dissolved in water/acetonitrile/acetic acid (e.g. 90/9/1 v/v/v) and loaded onto a HPLC column (loading up to 20 g/L, bed depth approx. 25 cm) and the purification program (a gradient from aqueous ammonium phosphate (approx. pH 2)/acetonitrile (80/20 v/v) to aqueous ammonium phosphate (approx. pH 2)/acetonitrile (60/40 v/v)) was initiated. Fractions were collected and diluted with water or diluted ammonium hydroxide solution.

### 2^{nd} Chromatography:

The pooled, diluted fractions from Chromatography 1 of [Aib^{8,35}]hGLP-1(7-36)NH₂ were loaded onto the HPLC column and the purification program (a gradient from aqueous ammonium acetate (approx. pH 9-10)/acetonitrile (85/15 v/v) to aqueous ammonium acetate (approx. pH 9-10)/acetonitrile (35/65 v/v)) was initiated. The fractions were collected and optionally diluted with water, diluted acetic acid or ammonium acetate. They were then loaded directly onto the HPLC column for the following concentration step.

### Concentration Step:

The pooled, diluted fractions from Chromatography 2 were loaded onto the same HPLC column and eluted quickly using a different mobile phase to concentrate peptide for isolation. As the mobile phase a gradient from aqueous ammonium acetate (approx. pH 9)/methanol (85/15 v/v) to aqueous ammonium acetate (approx. pH 9)/methanol (20/80 v/v) was used. The peptide containing fractions were pooled for isolation.

### Isolation:

In order to obtain a dry drug substance which is suitable for the drug formulation, the solution can either be subjected to precipitation, lyophilization or spray-drying techniques. Precipitation of the peptide:

The peptide solutions were slowly fed with isopropanol (IPA) at a temperature between 20° C. and 25° C. The mixture became cloudy. After stirring overnight at 20 to 25° C. the precipitating product [Aib^{8,35}]hGLP-1(7-36)NH₂ was filtered and washed with IPA and then dried at 25° C. under vacuum until a constant weight was obtained.

Alternatively, the peptide can be isolated by spray-drying as described in WO 2010/072621:

The peptide containing fraction were directly fed to a Niro SD-4-R-CC (Spraying chamber 0 1.2×0.75 m, capacity 8 kg H₂OZh). After a few hours, a fine powder of [Aib^{8,35}]hGLP-1(7-36)NH₂ was collected from the cyclone.

### Example 2

### Formulation Procedures

### A) Materials:

Zinc chloride, sodium chloride and 1M hydrochloric acid were purchased from Merck. Acetic acid (99.5%) was purchased from Fluka. Sodium acetate trihydrate was purchased from Hänseler AG. Water for injection was internally produced by double-destillation.

### B) Preparation of Stock Solutions:

Zinc Chloride: 5.236 g zinc chloride was dissolved in 100 mL water for injection.

Sodium Chloride: 5.844 g sodium chloride was dissolved in 100 mL water for injection.
Sodium acetate trihydrate: 10.478 g sodium acetate trihydrate was dissolved in 100 mL water for injection.

### C) Preparation of Liquid Formulations:

Approximately 70% of target volume water for injection was filled into a compounding container and then acidified with acidic acid, 1N hydrochloric acid or combination of both. In case of acidic acid/sodium acetate mixtures sodium acetate trihydrate stock solution was added to the solution. Subsequently, [Aib^{8,35}]hGLP-1(7-36)NH₂ was added under stirring to the acidified solution. After dissolution of all solid [Aib^{8,35}]hGLP-1(7-36)NH₂ zinc chloride and/or sodium chloride stock solutions were added to the formulations. After further stirring the pH was measured and adjusted, respectively, by addition of 1N hydrochloric acid. Finally, water for injection was added to the formulation until the target volume has been reached. The final bulk formulation was sterile-filtered using a 0.22 μm PVDF filter into a clean and sterile container. The sterile bulk formulation was filled into heat-sterilized 6 mL glass vials with a fill volume of 5 mL. The vials were stoppered with teflonized serum stoppers and crimped with aluminium caps. Sterile filtration and vial filling were performed under aseptic conditions using a laminar air-flow bench.

### Overview of Tested Compositions

| | | |
|---|---|---|
| [Aib^{8, 35}]hGLP-1(7-36)NH₂ | fixed | 100 mg/mL (30 mM) |
| (GLP-1 analogue) | | |
| Zinc chloride | fixed | 2.72 mg/mL (20 mM) |
| Acetate¹ | varied | 0 to 95 mM |
| pH | varied | 4.4 to 4.6 |
| Sodium chloride | varied | 0 to 50 mM |
| Water for Injection | fixed | Quantum satis ad volume |

¹provided as a mixtures of acetic acid and sodium acetate trihydrate or acetic acid and hydrochloric acid

TABLE 1
Tested compositions

| | Fixed | | | | |
|---|---|---|---|---|---|
| Comp. | Parameters | Acetate | | pH | NaCl |
| A | 100 mg/mL | 95 | mM acetic acid | 4.46 | 0 mM |
| B | GLP-1 analogue | 0 | mM acetic acid | 4.49 | 50 mM |
| | 2.72 mg/mL | Ad pH 1N HCl | | | |
| C | Zinc chloride | 47.5 | mM acetic acid | 4.51-4.55 | 25 mM |
| | Qs ad volume | Ad pH 1N HCl | | | |
| D | Water for | 75 | mM acetic acid | 4.49 | 50 mM |
| | injection | 20 | mM sodium | | |
| | | | acetate trihydrate | | |
| E | | 0 | mM acetic acid | 4.41 | 50 mM |
| | | Ad pH 1N HCl | | | |
| F | | 75 | mM acetic acid | 4.60 | 0 mM |
| | | 20 | mM sodium | | |
| | | | acetate trihydrate | | |
| G | | 0 | mM acetic acid | 4.42 | 0 mM |
| | | Ad pH 1N HCl | | | |
| H | | 95 | mM acetic acid | 4.54 | 50 mM |
| I | | 0 | mM acetic acid | 4.54 | 0 mM |
| | | Ad pH 1N HCl | | | |
| J | | 20 | mM acetic acid | 4.51 | 0 mM |

Ad pH 1N HCl

### Composition A (Original Formulation, Comparative Example)

100 mg/mL (30 mM) [Aib^{8,35}]hGLP-1(7-36)NH₂

95 mM Acetic Acid

2.72 mg/mL (20 mM) Zinc Chloride

pH 4.5

Water for Injection (quantum satis ad volume)

### Composition B

100 mg/mL (30 mM) [Aib^{8,35}]hGLP-1(7-36)NH₂

0 mM Acetic Acid

2.72 mg/mL (20 mM) Zinc Chloride

50 mM Sodium Chloride

Hydrochloric acid quantum satis ad pH 4.5

Water for Injection (quantum satis ad volume)

### Composition C

100 mg/mL (30 mM) [Aib^{8,35}]hGLP-1(7-36)NH₂

47.5 mM Acetic Acid

2.72 mg/mL (20 mM) Zinc Chloride

25 mM Sodium Chloride

Hydrochloric acid quantum satis ad pH 4.5

Water for Injection (quantum satis ad volume)

### Composition D

100 mg/mL (30 mM) [Aib^{8,35}]hGLP-1(7-36)NH₂

75 mM Acetic Acid

20 mM Sodium Acetate Trihydrate

2.72 mg/mL (20 mM) Zinc Chloride

50 mM Sodium Chloride

pH 4.5

Water for Injection (quantum satis ad volume)

### Composition E

100 mg/mL (30 mM) [Aib^{8,35}]hGLP-1(7-36)NH₂

0 mM Acetic Acid

2.72 mg/mL (20 mM) Zinc Chloride

50 mM Sodium Chloride

Hydrochloric acid quantum satis ad pH 4.4

Water for Injection (quantum satis ad volume)

### Composition F

100 mg/mL (30 mM) [Aib^{8,35}]hGLP-1(7-36)NH₂

75 mM Acetic Acid

20 mM Sodium Acetate Trihydrate

2.72 mg/mL (20 mM) Zinc Chloride

pH 4.60

Water for Injection (quantum satis ad volume)

### Composition G

100 mg/mL (30 mM) [Aib^{8,35}]hGLP-1(7-36)NH₂

0 mM Acetic Acid

2.72 mg/mL (20 mM) Zinc Chloride

Hydrochloric acid quantum satis ad pH 4.4

Water for Injection (quantum satis ad volume)

### Composition H

100 mg/mL (30 mM) [Aib^{8,35}]hGLP-1(7-36)NH₂

95 mM Acetic Acid

2.72 mg/mL (20 mM) Zinc Chloride

50 mM Sodium Chloride

pH 4.5

Water for Injection (quantum satis ad volume)

### Composition I

100 mg/mL (30 mM) [Aib^{8,35}]hGLP-1(7-36)NH₂

0 mM Acetic Acid

2.72 mg/mL (20 mM) Zinc Chloride

Hydrochloric acid quantum satis ad pH 4.5

Water for Injection (quantum satis ad volume)

### Composition J

100 mg/mL (30 mM) [Aib^{8,35}]hGLP-1(7-36)NH₂

20 mM Acetic Acid

2.72 mg/mL (20 mM) Zinc Chloride

Hydrochloric acid quantum satis ad pH 4.5

Water for Injection (quantum satis ad volume)

### Example 3

### Analytical Procedures

The impact of formulation modifications on the sustained release profile of [Aib^{8,35}]hGLP-1(7-36)NH₂ was investigated by using in vitro precipitation and dissolution assays. Furthermore, formulations of [Aib^{8,35}]hGLP-1(7-36)NH₂ were analyzed for buffer capacity, viscosity and turbidity.

### Example 3.1

### Precipitation Assay

The precipitation assay measures the remaining soluble active ingredient after precipitation of [Aib^{8,35}]hGLP-1(7-36)NH₂ formulation in a (USP) 50 mM phosphate buffer pH 7.4. 200 μl of the [Aib^{8,35}]hGLP-1(7-36)NH₂ formulation were mixed with 250 μl of phosphate buffer. The mixture was centrifuged and the clear supernatant was analyzed for the concentration of [Aib^{8,35}]hGLP-1(7-36)NH₂.

TABLE 2
Relative content of [Aib^{8,35}]hGLP-1(7-36)NH₂ in the
supernatant after precipitation in phosphate buffer pH 7.4 and
subsequent centrifugation.

| | Comp. | | | | | | | | | |
|---|---|---|---|---|---|---|---|---|---|---|
| | A | B | C | D | E | F | G | H | I | J |
| % dose in | 15.8 | 0.2 | 0.5 | 2.3 | 0.3 | 2.3 | 0.6 | 2.4 | 0.2 | 0.3 |
| supernatant | | | | | | | | | | |

Modified formulations showed a significant reduction of [Aib^{8,35}]hGLP-1(7-36)NH₂ solubility in phosphate buffer pH 7.4 in comparison of the original formulation (A).

### Example 3.2

### Dissolution Assay in HBSS Medium

The dissolution assay measures the release of [Aib^{8,35}]hGLP-1(7-36)NH₂ from a precipitated depot to a continuous flow of release medium as a function of time. 200 μl of the [Aib^{8,35}]hGLP-1(7-36)NH₂ formulation were added to a USP IV powder cell filled with glass beadles. The dissolution assay was run with 2 mL/min for 60 min in the open configuration. Samples were collected after 5, 10, 15, 30 and 60 min. The release medium was a modified Hanks balanced salt solution (HBSS) with 25 mM HEPES at pH 7.4 as buffering agent according to Iyer et al., J. Pharmaceut. Biomed. Anal. (2006), pages 119-125, Characterization of a potential medium for “biorelevant” in vitro release testing of a naltrexone implant, employing a validated stability-indicating HPLC method).

TABLE 3
Released amount of [Aib^{8,35}]hGLP-1(7-36)NH₂ per time in mg/min during
dissolution in modified HBSS buffer pH 7.4.

| | Comp. | | | | | | | | | |
|---|---|---|---|---|---|---|---|---|---|---|
| | A | B | C | D | E | F | G | H | I | J |
| mg/min | 0.054 | 0.001 | 0.006 | 0.005 | 0.004 | 0.009 | 0.006 | 0.004 | 0.011 | 0.000 |
| dissolved | | | | | | | | | | |
| after 5 min | | | | | | | | | | |
| mg/min | 0.058 | 0.006 | 0.007 | 0.016 | 0.008 | 0.006 | 0.012 | 0.009 | 0.017 | 0.006 |
| dissolved | | | | | | | | | | |
| after 10 min | | | | | | | | | | |
| mg/min | 0.059 | 0.009 | 0.010 | 0.020 | 0.008 | 0.010 | 0.014 | 0.014 | 0.014 | 0.010 |
| dissolved | | | | | | | | | | |
| after 15 min | | | | | | | | | | |
| mg/min | 0.059 | 0.011 | 0.015 | 0.021 | 0.008 | 0.021 | 0.014 | 0.023 | 0.010 | 0.011 |
| dissolved | | | | | | | | | | |
| after 30 min | | | | | | | | | | |
| mg/min | 0.047 | 0.016 | 0.017 | 0.026 | 0.006 | 0.020 | 0.012 | 0.028 | 0.011 | 0.013 |
| dissolved | | | | | | | | | | |
| after 60 min | | | | | | | | | | |

Modified formulations showed a significant reduction of dissolution of [Aib^{8,35}]hGLP-1(7-36)NH₂ in modified HBSS pH 7.4 in comparison to the original formulation (A).

### Example 3.3

### Determination of Buffer Capacity

The buffer capacity assay determines the amount of NaOH needed to titrate a defined amount of [Aib^{8,35}]hGLP-1(7-36)NH₂ formulation to a pH of 7.4.

5 mL of the [Aib^{8,35}]hGLP-1(7-36)NH₂ formulation was filled into 20 mL glass beaker. 0.1M sodium hydroxide was added under continuous stirring to the solution. The pH was recorded potentiometrically using a glass electrode until a pH of 7.4 is reached. The buffer capacity is reported as mmol sodium hydroxide per L test solution.

TABLE 4
Buffer capacity of [Aib^{8,35}]hGLP-1(7-36)NH₂ formulations:

| | Comp. | | | | | | | | | |
|---|---|---|---|---|---|---|---|---|---|---|
| | A | B | C | D | E | F | G | H | I | J |
| mmol/L | 107 | 64 | 82 | 114 | 70 | 99 | 69 | 99 | 59 | 62 |
| NaOH to | | | | | | | | | | |
| reach pH | | | | | | | | | | |
| 7.4 | | | | | | | | | | |

Modified formulations B, C, E, G, I and J showed a clear reduction of buffer capacity in comparison to the original formulation (A) and formulations D and H.

### Example 3.4

### Determination of Viscosity

The viscosity was determined on a plate and cone rheometer at a shear rate of 2000 s⁻¹ and at a temperature of 20° C. The viscosity is a relevant parameter to assess the syringe ability of a formulation through a needle for subcutaneous delivery and a viscosity below 10 mPas is desirable for a very easy manual injection (W. Rungseevijitprapa and R. Bodmeier; Eur. J. Pharm. Sci. 36 (2009), 524-531. Injectability of biodegradable in situ forming microparticle systems (ISM)).

TABLE 5
Viscosity of [Aib^{8,35}]hGLP-1(7-36)NH₂ formulations
determined on a plate and cone rheometer at a shear rate of
2000 s⁻¹ and a temperature of 20° C.

| | Comp. | | | | | | | | | |
|---|---|---|---|---|---|---|---|---|---|---|
| | A | B | C | D | E | F | G | H | I | J |
| Viscosity | 3.5 | 10.3 | 4.7 | 11.4 | 6.8 | 4.3 | 3.5 | 9.8 | 3.7 | 3.3 |
| in mPas | | | | | | | | | | |

The viscosity of formulations A, C, F, G, I and J was clearly lower than determined for formulations B, D, E and H. The presence of 50 mM sodium chloride in the formulations B, D, E and H led to a clear increase of viscosity.

### Example 3.5

### Determination of Turbidity

The turbidity of the [Aib^{8,35}]hGLP-1(7-36)NH₂ formulations was measured on a Hach 2199 AN turbid meter. The turbidity indicates the physical stability of a liquid solution and an elevated turbidity points to a decreased solubility of [Aib^{8,35}]hGLP-1(7-36)NH₂ and the risk of potential precipitation.

TABLE 6
Turbidity of [Aib^{8,35}]hGLP-1(7-36)NH₂ formulations

| | Comp. | | | | | | | | | |
|---|---|---|---|---|---|---|---|---|---|---|
| | A | B | C | D | E | F | G | H | I | J |
| Turbidity | 4.6 | 23.5 | 10.3 | 29.5 | 15.5 | 7.8 | 4.0 | 24.9 | 5.2 | 4.1 |
| in FTU | | | | | | | | | | |

The turbidity of formulations A, C, F, G, I and J is clearly lower than determined for the formulations B, D, E and H. The presence of 50 mM sodium chloride in the formulations B, D, E and H led to a clear increase of turbidity.

1. A sustained release liquid pharmaceutical composition having a pH in the range of 4.4 to 4.6 comprising:
a liquid solvent,
a peptide analogue having the formula
[Aib^{8,35}]hGLP-1(7-36)NH₂(I), and
a divalent metal salt, wherein the molar ratio range of the peptide analogue to the divalent metal is 1.5 to 1,
characterized in that the final pH in the range of 4.4 to 4.6 is adjusted by addition of hydrochloric acid.

2. A pharmaceutical composition according to claim 1, wherein the liquid solvent is water.

3. A pharmaceutical composition according to claim 1, wherein the divalent metal salt is zinc chloride.

4. A pharmaceutical composition according to claim 3, wherein the concentration of zinc chloride is 2.72 mg/ml (20 mM).

5. A pharmaceutical composition according to claim 1, wherein the concentration of the peptide analogue is 100 mg/ml (30 mM).

6. A pharmaceutical composition according to claim 1, wherein the composition further comprises an acetic acid and/or an acetate salt, wherein the molar ratio range of the acetic acid and/or acetate salt to the peptide is less than 3.2 to 1.

7. A pharmaceutical composition according to claim 6, wherein the maximum concentration of acetic acid and/or sodium acetate trihydrate is 95 mM.

8. A pharmaceutical composition according to claim 6 comprising:
water,
[Aib^{8,35}]hGLP-1(7-36)NH₂ in a concentration of 100 mg/ml (30 mM)
zinc chloride in a concentration of 2.72 mg/ml (20 mM), and
acetic acid and/or sodium acetate trihydrate, wherein acetic acid is added in a concentration range of 0 mM to 75 mM,
characterized in that the final pH in the range of 4.4 to 4.6 is adjusted by addition of hydrochloric acid.

9. A pharmaceutical composition according to claim 8, wherein acetic acid is added in a concentration range of 0 mM to 47.5 mM.

10. A pharmaceutical composition according to claim 9, wherein acetic acid is added in a concentration of 20 mM.

11. A pharmaceutical composition according to claim 1, wherein the composition is stable at a temperature of 5° C. for a period of at least one year.

12. A pharmaceutical composition according to claim 1, wherein the composition is formulated such that the peptide analogue of formula (I) is released within the subject for at least approximately 1 week.

13. A pharmaceutical composition according to claim 1, wherein the composition is formulated such that the peptide analogue of formula (I) is released within the subject for at least approximately 2 weeks

14. A pharmaceutical composition according to claim 1, wherein less than 2.5% of the dose of the peptide analogue of formula I can be detected in the supernatant after precipitation in phosphate buffer pH 7.4 and subsequent centrifugation.

15. A pharmaceutical composition according to claim 1, wherein less than 0.015 mg/min of the peptide analogue of formula I are released after 5 min during dissolution in modified HBSS buffer pH 7.4.

16. A pharmaceutical composition according to claim 1, wherein said composition is kept in a container.

17. A pharmaceutical composition according to claim 16, wherein said container is a pre-filled syringe.

18. A pre-filled syringe containing a pharmaceutical composition according to claim 1.
